(19) United States
(12) Patent Application Publication
(10) Pub. No.: US 2018/0280322 A1
(43) Pub. Date: Oct. 4, 2018

(54) STABLE FORMULATIONS OF FINGOLIMOD

(71) Applicant: Mylan Inc., Canonsburg, PA (US)

(72) Inventors: Sarat C Chattaraj, Morgantown, WV (US); Zhi Liu, Morgantown, WV (US); John Kirsch, Waynesburg, PA (US)

(73) Assignee: Mylan Inc., Canonsburg, PA (US)

(21) Appl. No.: 15/764,169

(22) PCT Filed: Aug. 8, 2016
(86) PCT No.: PCT/US16/46002

§ 371 Date: Mar. 28, 2018

Related U.S. Application Data
(60) Provisional application No. 62/236,373, filed on Oct. 2, 2015.

(51) Int. Cl. A61K 31/137; A61K 9/48

(52) U.S. Cl. CPC A61K 31/137; A61K 9/485; A61K 9/4858

(57) ABSTRACT

Sugar alcohol-free formulations of fingolimod. Compositions of fingolimod, salts thereof, or esters thereof that lack sugar alcohols are disclosed. The composition may include a water-soluble filler and a water-insoluble filler, in addition to other common excipients. In some examples, the water-soluble filler is glycine and the water-insoluble filler is dibasic calcium phosphate dihydrate as fillers. In some examples, the water-soluble filler and water-insoluble filler are present in equal concentrations. The compositions disclosed here may be used to make immediate release dosage forms containing fingolimod hydrochloride.

### CROSS-REFERENCE TO RELATED APPLICATIONS

This application claims the benefit of U.S. provisional patent application No. 62/236,373 filed on Oct. 2, 2015.

### BACKGROUND OF THE INVENTION

### 1. Field of the Invention

The present invention relates generally to the field of pharmaceutical formulations, and more specifically to a formulation of fingolimod.

### 2. Description of the Background

Fingolimod is an immunomodulatory drug that modulates the sphingosine-1-phospate receptor resulting in the sequestration of lymphocytes in lymph nodes. Fingolimod's chemical structure is shown below:

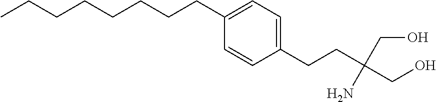 [SMILES: CCCCCCCCc1ccc(CCC(N)(CO)CO)cc1]

Chemically, it is known as 2-amino-2-[2-(4-octylphenyl)ethyl]propan-1,3-diol hydrochloride.

Fingolimod hydrochloride (HCl) capsules are currently marketed by Novartis under the trade name GILENYA®. GILENYA® is indicated for the treatment of multiple sclerosis (MS) to reduce the frequency of clinical exacerbations and to delay the accumulation of physical disability. Commercial formulation of fingolimod (e.g., GILENYA®) include mannitol as a filler. Fingolimod HCl is known to react with that mannitol filler, which may lead to loss of quality and efficacy during prolonged storage of the dosage form See Page 2, WO 2014/013090, which is hereby incorporated by reference. To mitigate these issues, storage of GILENYA® below 30° C. is recommended.

Further, it was well known in the pharmaceutical arts that compounds that include an amine group are commonly subject to degradation through a chemical process known as a Malliard Reaction. The Malliard Reaction occurs between an amine-containing compound and a reducing sugar (e.g., sucrose, lactose, and fructose). To avoid the deleterious effects of the Malliard Reaction, the prior art would employ a non-reducing sugar (e.g., mannitol), rather than a reducing sugar. In the case of fingolimod HCl, such traditional approaches are not satisfactory because of the known interaction between mannitol and fingolimod HCl.

The prior art developed formulations of fingolimod that were sugar-alcohol (including mannitol)-free, including WO2014/013090 and U.S. Patent App. Pub. No. 2014/0199382, which are hereby incorporated by reference.

Glycine is a known stabilizer utilized in pharmaceutical formulations (see, e.g., published U.S. Patent App. Pub. No. 2014/0255497, U.S. Pat. No. 2,649,993, and PCT App. No. PCT/US1995/002452 A1, which are hereby incorporated by reference). Glycine, however, is not commonly employed as a filler in pharmaceutical formulations.

A common challenge in developing pharmaceutical formulations is identifying combinations of excipients that, when combined, result in a pharmaceutical formulation having good workability. At the same time, excipients and active pharmaceutical ingredient (API) should be physically and chemically stable. Further, the combination of excipients and API in the pharmaceutical formulation should achieve the desired release characteristics.

With these general goals in mind, the problem confronting the art is to develop a new formulation of fingolimod hydrochloride that does not contain mannitol as a filler. At the same time, the desired composition uniformity and workability, formulation and API stability profile, and immediate release characteristics of the formulation are maintained or improved.

### SUMMARY OF THE INVENTION

The present invention provides compositions, and methods of their formulation, that include fingolimod or a pharmaceutically acceptable salt or ester thereof as an active agent and where the composition lacks a sugar alcohol. In some embodiments, the composition employs dicalcium basic phosphate dihydrate and glycine together as fillers. The compositions of the present invention possess good workability and uniformity and may be employed in the formulation of solid dosage pharmaceutical forms.

In some embodiments of the present invention, a pharmaceutical dosage form is provided that includes a water-soluble filler, a water-insoluble filler, and fingolimod, which may be present as fingolimod HCl. The pharmaceutical dosage form preferably does not include a sugar alcohol such as mannitol. The water-soluble filler may be selected from the group of glycine, arginine, cysteine hydrochloride, methionine, and sodium chloride. The water-insoluble filler may be an inorganic salt such as, for example, dibasic calcium phosphate, tribasic calcium phosphate, calcium sulfate, and any anhydrous or hydrated form thereof. In certain embodiments, the water-soluble filler is glycine and the water-insoluble filler is dibasic calcium phosphate dihydrate.

In some embodiments, the concentration of glycine is approximately equal to the concentration of dibasic calcium phosphate dihydrate, by weight. In some embodiments, both the glycine and dibasic calcium phosphate dihydrate are present at a concentration about 35% to about 49%, by weight.

In some embodiments, both the glycine and dibasic calcium phosphate dihydrate are present at a concentration about 35% to 85% glycine and 13% to 63% dibasic calcium phosphate dihydrate by weight.

In some embodiments, glycine is present at a concentration about 35% to 95%9 glycine and 3% to 63% dicalcium basic phosphate dihydrate by weight.

In some embodiments, glycine is present at a concentration about 5% to about 95% and dicalcium phosphate is present at a concentration about 5% to about 95%.

In some embodiments, the pharmaceutical dosage form may also include a lubricant and a glidant. The lubricant may be magnesium stearate, magnesium stearate with sodium lauryl sulfate (94:6), sodium stearyl fumarate, Compritol® 888 ATO, or calcium stearate. The glidant may be colloidal silicon dioxide.

The pharmaceutical dosage forms of the present invention include between about 0.1 to about 10 milligrams of fingolimod HCl per dosage form. The pharmaceutical dosage forms of the present invention may be formulated through the use of mixing, dry granulation, wet granulation, or granulation by extrusion.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 shows the in vitro drug release profiles of a fingolimod capsule of the present invention compared to the reference listed drug product GILENYA®;

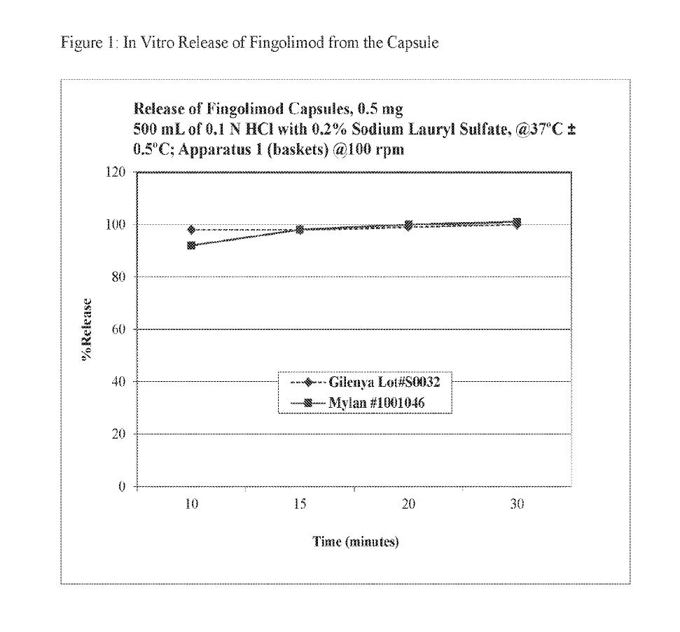

FIG. 2 displays a fasting state pharmacokinetic profile as a graph (linear Scale) of in-vivo mean blood concentration of fingolimod versus time for a capsule (single dose, 1×0 5 mg) of the present invention (where in the capsule contains fingolimod hydrochloride) as compared to GILENYA®; and

FIG. 3 displays a fasting state pharmacokinetic profile as a graph (semi-log scale) of in-vivo mean blood concentration of fingolimod versus time for a capsule (single dose, 1×0.5 mg) of the present invention (where in the capsule contains fingolimod hydrochloride) as compared to GILENYA®, and

FIG. 4 displays a fasting state pharmacokinetic profile as a graph (linear Scale) of in-vivo mean blood concentration versus time of fingolimod phosphate for a capsule (single dose, 1×0.5 mg) of the present invention (where in the capsule contains fingolimod hydrochloride) as compared to GILENYA®; and

FIG. 5 displays a fasting state pharmacokinetic profile as a graph (semi-log scale) of in-vivo mean blood concentration versus time of fingolimod phosphate for a capsule (single dose, 1×0.5 mg) of the present invention (where in the capsule contains fingolimod hydrochloride) as compared to GILENYA®; and

FIG. 6 shows a fed state pharmacokinetic profile as a graph (linear Scale) of in-vivo mean blood concentration of fingolimod versus time for a capsule (single dose, 1×0.5 mg) of the present invention wherein the capsule contains fingolimod hydrochloride as compared to GILENYA®; and

FIG. 7 shows a fed state pharmacokinetic profile as a graph (semi-log scale) of in-vivo mean blood concentration of fingolimod versus time for a capsule (single dose, 1×0.5 mg) of the present invention wherein the capsule contains fingolimod hydrochloride as compared to GILENYA®; and

FIG. 8 displays a fed state pharmacokinetic profile as a graph (linear Scale) of in-vivo mean blood concentration of fingolimod phosphate versus time for a capsule (single dose, 1×0.5 mg) of the present invention (where in the capsule contains fingolimod hydrochloride) as compared to GILENYA®, and

FIG. 9 displays a fed state pharmacokinetic profile as a graph (semi-log scale) of in-vivo mean blood concentration of fingolimod phosphate versus time for a capsule (single dose, 1×0.5 mg) of the present invention (where in the capsule contains fingolimod hydrochloride) as compared to GILENYA®.

### DETAILED DESCRIPTION OF THE INVENTION

It is to be understood that the figures and descriptions of the present invention have been simplified to illustrate elements that are relevant for a clear understanding of the invention, while eliminating for purposes of clarity, other elements that may be well known. The detailed description will be provided herein below with reference to the attached drawing.

As noted above, one of the challenges facing the art was to formulate fingolimod hydrochloride (or other pharmaceutically acceptable salts or esters thereof) as a solid dosage form without using sugar alcohols, which destabilize fingolimod-containing formulations. At the same time the compositions should possess good uniformity and workability. As used herein, workability describes a formulation with good flow properties and robustness that is easy to manufacture with standard equipment and without complex processes.

To develop such a new formulation of fingolimod hydrochloride, researchers were confronted with a daunting task. One of the most commonly employed class of pharmaceutical fillers is sugar alcohols (e.g., mannitol, xylitol). The properties of these components are well understood and thoroughly investigated, but their use in pharmaceutical formulations containing fingolimod is inappropriate.

The inventors here arrived at the formulations disclosed herein after many failed attempts to create a fingolimod composition that did not include sugar alcohols. For example, inventors attempted formulations that substituted starches (e.g., pre-gelatinized starches) for mannitol as the primary filler; however, potency of the final formulation decreased as a result. In other attempts, the filler included both dibasic calcium phosphate dihydrate and microcrystalline cellulose (e.g., AVICEL PH 101), however the stability of this formulation was less than desired, as was the purity. Further attempts utilized dibasic calcium phosphate dihydrate as the only primary filler, however the dissolution rate of the capsule was slower than desired. Yet other failed attempts utilized combinations of dibasic calcium phosphate dihydrate with crospovidone CI. M and dibasic calcium phosphate dihydrate with crospovidone XL; these attempts failed due to decreased product stability.

In continuing efforts, researchers looked to employ other components as non-traditional fillers. The researchers finally turned to using glycine as a filler. As noted above, glycine is commonly employed in pharmaceutical formulations as a stabilizer, but not as a filler. Further, it was unknown whether such high concentrations of glycine would interact with either dibasic calcium phosphate dihydrate or with the API fingolimod.

Surprisingly, even though glycine is not typically employed as a filler, the presently disclosed compositions possess desirable properties for a pharmaceutical formulation, including good uniformity and workability. Further, the fingolimod formulations of the present invention are stable, as investigated in accelerated stability testing, which is described further herein below. Finally, the present compositions may be used in pharmaceutical formulations to achieve desired immediate-release characteristics for a fingolimod-containing pharmaceutical formulation.

The present invention provides a solid composition containing fingolimod, a pharmaceutically acceptable salt of fingolimod, or an ester of fingolimod suitable for incorporation into pharmaceutical dosage forms. In some embodiments, the formulations of the present invention contain fingolimod hydrochloride, a water-soluble filler, and a water-insoluble filler.

Fingolimod may be included in the present invention in any pharmaceutically acceptable form. This includes pharmaceutically acceptable salts of fingolimod or esters of fingolimod. In some embodiments of the present invention, fingolimod hydrochloride is employed. Fingolimod may be included in the composition at any concentration appropriate for the final pharmaceutical formulation. In some embodiments, fingolimod hydrochloride is present at a concentration of 0.5 milligrams per dosage form, though concentrations ranging from 0.1 to 10 milligrams per dosage form may also be used.

The formulations of the present invention include a water-soluble filler Examples of suitable water-soluble fillers include glycine, arginine, cysteine hydrochloride, methionine, and sodium chloride. In particularly useful embodiments of the present invention, glycine is employed as the water-soluble filler.

The formulations of the present invention further include a water-insoluble filler. Within the context of the present invention, suitable water-insoluble fillers include dibasic calcium phosphate, tribasic calcium phosphate, calcium sulfate, and all solvated and anhydrous forms thereof. In particularly useful embodiments of the present invention, the water-insoluble filler is dibasic calcium phosphate dihydrate.

The concentration of the water-soluble or water-insoluble filler may range from about 35% to about 49%, by weight, of the composition. In particularly useful embodiments, the water-soluble filler and the water-insoluble filler are included at substantially equal concentrations, at approximately 1:1 ratio of concentration of water-soluble filler to water-insoluble filler. In some embodiments, the water-soluble filler and water-insoluble filler are each present at about 49% by weight of the entire composition. In some embodiments, a composition of the present invention includes 47.7% water-soluble filler and 47.7% water insoluble filler.

Another aspect of the present invention provides a method of preparation of the solid formulation of fingolimod. The compositions described above may be in the form of powder, granule, mini-tablets, pellets, or a unit dosage form (e.g., tablet, capsule) or a mixture of powder with granule or mini-tablets or pellets or tablet. Formulation into a unit dosage form may be accomplished by convention mixing, dry granulation, wet granulation, granulation by extrusion and other methods well known to those of skill in the art. A wet granulation approach was evaluated initially; however, this approach was abandoned after few experiments due to the concern of polymorphism change of the drug due to the addition of water and drying process. The poor flow properties of the drug, and sensitivities to certain excipients, combined with the low dose of drug, posed a challenge to achieve content uniformity and stability of the finished product. A workable formulation was achieved by dry granulation (Blend-Compact/Fitz-mill/Blend/Final Blend/Encapsulation) based process which produced a well-flowing uniform granular blend. The formulation prepared by these methods carries the drug in uniform distribution and allowed for good weight control during encapsulation without any issue. The fingolimod capsules prepared with these materials and by these methods demonstrated a good immediate release profile and performed well when exposed to the accelerated conditions of stability.

In some embodiments, a dry granulation and milling process may be undertaken through the following steps:
1. Combining the fingolimod, a pharmaceutically acceptable salt of fingolimod, or an ester of fingolimod with excipients to create a mixture;
2. Passing the mixture through a roller compactor;
3. Milling the mixture; and
4. Blending the mixture with extragranular excipients.

The excipients may include a wide variety of components such as fillers, lubricants, coloring agents, flavoring agents, glidants, and preservatives. Within the context of the present invention, the fillers include water-soluble fillers, water-insoluble fillers, and mixtures thereof. In a particularly effective embodiment, the fillers are glycine and dibasic calcium phosphate dihydrate. Such excipients may be combined with fingolimod (or a pharmaceutically acceptable salt or ester thereof) to create a solid mixture

Next, the mixture is roller compacted and milled to achieve granules. In a particularly useful embodiment of the invention, milling is achieved by a Fitzmill. Next, the mixture is blended with additional extragranular excipients to create a final pharmaceutical blend. The mixture may be blended, for example, in a “V” blender. Suitable extragranular excipients include fillers, lubricants, coloring agents, flavoring agents, glidants, and preservatives.

After mixing and blending, the final pharmaceutical blend may be incorporated into a final pharmaceutical dosage form, for example, granules, tablets, and capsules. A lubricant such as magnesium stearate, magnesium stearate with sodium lauryl sulfate (94:6), sodium stearyl fumarate, Compritol® 888 ATO, stearic acid, or calcium stearate may additionally be added during manufacture of the final dosage form. In some embodiments, magnesium stearate is a particularly useful lubricant. This final dosage form may also have a coating or a shell that may include ingredients such as titanium dioxide, yellow iron oxide, and gelatin.

In view of the above description and the examples below, one of ordinary skill in the art will be able to practice the invention as claimed without undue experimentation. The foregoing will be better understood with reference to the following examples that detail certain procedures for the preparation of molecules, compositions and formulations according to the present invention. All references made to these examples are for the purposes of illustration. The following examples should not be considered exhaustive, but merely illustrative of only a few of the many aspects and embodiments contemplated by the present disclosure.

### Example 1

Formulation of a unit dosage form may be carried out by (i) dry mixing the glycine, dibasic dicalcium phosphate dihydrate, colloidal silicon dioxide and magnesium stearate in a “V” blender for 10 minutes; (ii) adding fingolimod hydrochloride, dibasic calcium phosphate dihydrate to the same “V” blender; (iii) rinsing the container with a portion of the dibasic dicalcium phosphate dihydrate to remove any residual amount of fingolimod hydrochloride remaining in the container and adding the rinsed solution to the same “V” blender with additional glycine; (iv) blending that composition for 10 minutes; (v) incorporating additional glycine and dibasic calcium phosphate dihydrate to the above blender; (vi) blending for that mixture for 15 minutes.

Following blending of that composition the next steps are (vii) compacting the blended material using a Model L89 Compactor and milled using a Fitzmill, using a #1B screen (which preferably has openings of about 1.27 mm), blade position: knife forward and blade speed: medium, (viii) adding the compacted/milled material to a “V” blender; (ix) blending for 25 minutes; (x) sampling this composition for blend uniformity testing; (xi) assigning a potency factor for the composition; (xii) adding final various excipients to the “V” blender to achieve desired final concentrations; (xiii) blending the composition for 10 minutes in the “V” blender; (xiv) encapsulating the final blend using an MG encapsulation machine. The finished capsule products were packaged in blister packs (base film: Aclar®, PVC, and push-through foil) and in sealed bottles. All the data presented herein is based on the blister-packed capsules.

### Example 2

Dosage forms as obtained in Example 1 were used to assess stability characteristics of formulations of the present invention. Specifically, a stress testing (i.e., forced degradation) study was conducted on the fingolimod formulation that utilized a mixture of equal parts glycine and dibasic calcium phosphate dihydrate as filler. As shown in the table below, this formulation demonstrated that the fingolimod hydrochloride active drug substance is very stable in the solid state in this formulation under stress conditions. The same table below shows the stability characteristics of fingolimod hydrochloride present in the other formulations, such as, GILENYA®, fingolimod hydrochloride and mannitol (1A1), fingolimod hydrochloride and dibasic calcium hydrate with microcrystalline cellulose (7A1), and fingolimod hydrochloride and dibasic calcium hydrate with crospovidone (19A1).

TABLE 1
Initial Stability Screening of Fingolimod Capsules, 0.5 mg

| | Formulation | | Acetyl | |
|---|---|---|---|---|
| Batch No. | Composition | Conditions | Amino Diol | Total Impurities |
| S0033 | GILENYA ® | 4 Weeks 40° C./75% RH | 0.23% | 0.49% |
| | | 8 Weeks 40° C./75% RH | 0.38% | 0.88% |
| | | 12 Weeks 40° C./75% RH | 0.52% | 1.00% |
| X13-027-1A1 | Mannitol | 4 Weeks 40° C./75% RH | 0.08% | 0.41% |
| | | 3 Days 70° C. | 0.13% | 0.98% |
| X13-027-7A1 | Dibasic calcium | 4 Weeks 40° C./75% RH | 0.18% | 1.51% |
| | phosphate dihydrate and | 3 Days 70° C. | 0.11% | 1.12% |
| | microcrystalline | | | |
| | cellulose (Avicel ®) | | | |
| X13-027-15A2 | Dibasic calcium | 4 Weeks 40° C./75% RH | 0.05% | 0.26% |
| | phosphate dihydrate and | 3 Days 70° C. | 0.07% | 0.66% |
| | glycine | | | |
| X13-027-19A1 | Dibasic calcium | 3 Days 70° C. | 0.05% | 1.42% |
| | phosphate dihydrate and | | | |
| | crospovidone | | | |

In Table 1, formulations 1A1 (with mannitol), 7A1 (with dibasic calcium phosphate dihydrate and microcrystalline cellulose), and 19A1 (with dibasic calcium phosphate dihydrate and crospovidone) exhibited a faster accumulation of total impurities under stress conditions when compared to the 15A2 (with dibasic calcium phosphate dihydrate and glycine) formulation. The total unknown impurities of 15A2 formulation were less than the reference product GILENYA® and the 1A1 formulation containing mannitol. Based on this study, the 15A2 formulation using dibasic calcium phosphate dihydrate and glycine was selected for further development.

### Example 3

Three batches of fingolimod hydrochloride capsules, 0.5 mg (Lot. No. 1001046, 1001060 and 1001061) were manufactured in large scale (batch size: 275,000 capsules each) using two different lots of fingolimod hydrochloride active drug substances. As shown in the table below, the total weight for each capsule is 75 mg.

TABLE 2
Fingolimod Hydrochloride Capsules, 0.5 mg
(Lot. Nos. 1001046, 1001060 and 1001061)

| Ingredients | mg/unit |
|---|---|
| PART I | |
| Dibasic calcium phosphate dihydrate (Emcompress ®) | 16.535 |
| Glycine | 16.535 |
| Colloidal silicon dioxide (Cab-O-Sil, M5P) | 0.1875 |
| Magnesium stearate | 0.1875 |
| PART II | |
| Fingolimod hydrochloride | 0.56* |
| Dibasic calcium phosphate dihydrate (Emcompress ®) | 11.16 |
| Glycine | 11.16 |
| PART III | |
| Dibasic calcium phosphate dihydrate (Emcompress ®) | 3.591 |
| Glycine | 3.591 |
| Total Theoretical Weight - Part I-III | 63.507 |
| PART IV | |
| Dibasic calcium phosphate dihydrate (Emcompress ®) | 5.484 |
| Glycine | 5.484 |
| Magnesium stearate | 0.3375 |
| Colloidal silicon dioxide (Cab-O-Sil, M5P) | 0.1875 |
| Total Theoretical Weight - Part IV | 11.493 |
| Total Theoretical Weight | 75.0 |

*0.56 mg fingolimod hydrochloride is equivalent to 0.5 mg fingotimod.

In Step #1, a lubrication pre-blend was made by mixing the Part I dibasic calcium phosphate dihydrate (Emcompress®), the Part I colloidal silicon dioxide (Cab-O-Sil, M5P), the Part I magnesium stearate and the Part I glycine were added to a “V” Blender and blended for ten (10) minutes to produce the Part I lubricant pre-blend. The Part I lubricant pre-blend was screened through a #18 mesh screen into a drum containing double poly-liners.

In the Step #2 (Blending), the Part I screened lubricant pre-blend was added to a “V” blender. The Part II dibasic calcium phosphate dihydrate (Emcompress®) and the Part II glycine were screened through a #18 mesh screen and into weighed, labeled drums containing double poly-liners. The Part 11 Fingolimod hydrochloride was added to the one “V” Blender. The container was rinsed by placing a portion of the Part II screened dibasic calcium phosphate dihydrate (Emcompress®) into the container. The rinse material was added to the same “V” blender. The remaining Part II screened dibasic calcium phosphate dihydrate (Emcompress®) and Part II screened glycine were added to the same “V” blender and blended for ten (10) minutes. The Part III dibasic calcium phosphate dihydrate (Emcompress®) and the Part II glycine were screened through a #18 mesh screen were added to the same “V” Blender and blended for fifteen (15) minutes to produce the Part I-III blended material.

In step #3 (Compacting/Milling/Blending), the Part I-III material was roller compacted/milled and was added to a “V” blender and blended for twenty-five (25) minutes to produce the Part I-III second blended material.

In step #4 (Lubricant Pre-blending), the Part IV calculated quantities of dibasic calcium phosphate dihydrate (Emcompress®), colloidal silicon dioxide (Cab-O-Sil, M5P), magnesium stearate and the glycine were added to a “V” blender and blended for ten (10) minutes to produce the Part IV lubricant pre-blend. The Part IV screened lubricant pre-blend was screened through a #18 mesh screen and into weighed, labeled drums containing double poly-liners.

In step #7 (Final Blending), approximately one-half (½) of the Part 1-III second blended material, the Part IV screened lubricant pre-blend and the remaining Part I-III second blended material were added to a “V” Blender and blended for fifteen (15) minutes to produce the final blended material.

In step #8 (encapsulation), the final blended material was encapsulated using an MG encapsulation machine to a target fill weight of 75 mg using a size #3 hard gelatin capsule.

The finished capsules were packaged in unit dose blister packs (base film: Aclar®; push-through foil).

### Example 4

The following table presents the physical characteristics of final blend material of fingolimod hydrochloride capsules obtained from the three different lots. The Haussner's ratio was determined using formula [Haussner ratio=(tapped density/bulk density)]. The bulk density was determined by pouring a known quantity of blend material into a measuring cylinder and measuring the volume. The bulk density was calculated using the formula (bulk density=mass of the sample blend material/bulk volume of the sample blend material). In addition to the Haussner ratio, the compressibility index was also determined. The compressibility index was determined by the formula [compressibility index=(tapped density-bulk density)/(tapped density)×100].

TABLE 3
Physical properties of fingolimod capsules, 0.5 mg (final blend)

| | Lot#1001046 | Lot#1001060 | Lot#1001061 |
|---|---|---|---|
| Bulk density (g/ml) | 0.97 | 0.94 | 1.24 |
| Tapped density (g/ml), ×200 | 1.22 | 1.18 | 1.34 |
| Haussner Ratio | 1.26 | 1.26 | 1.08 |
| Compressibility Index (%) | 20.5 | 20.3 | 7.5 |
| | Amount | Amount | Amount |
| | Retain | Retain | Retain |
| 20 Mesh | 0.0% | 0.0% | 1.2% |
| 40 Mesh | 4.8% | 4.8% | 4.1% |
| 60 Mesh | 15.5% | 14.3% | 16.0% |
| 80 Mesh | 19.8% | 21.4% | 23.4% |
| 100 Mesh | 11.9% | 18.3% | 16.8% |
| 140 Mesh | 15.9% | 16.7% | 13.9% |
| Pan | 32.1% | 24.6% | 24.6% |

Table 4 presents the unit dose assay results of blend samples taken at various locations in the blend (top left, top center left, top center, top center right, top right, middle left, middle center, middle right; bottom left and bottom right). This confirms that the compaction/milling/blending process is robust.

TABLE 4
Assay Results of Unit Dose Blend Samples
of Fingolimod Capsules, 0.5 mg

| | | Lot#1001046 | Lot#1001060 | Lot#1001061 |
|---|---|---|---|---|
| Second Blend | Mean | 101.7% | 102.6% | 99.1% |
| | RSD | 0.9% | 2.5% | 0.7% |
| Final Blend | Mean | 102.3% | 100.2% | 99.9% |
| | RSD | 1.2% | 2.3% | 2.1% |

The content uniformity of the three lots are summarized in Table 5.

TABLE 5
Uniformity of dosage units of Fingolimod Capsules, 0.5 mg

| | Lot#1001046 | Lot#1001060 | Lot#1001061 |
|---|---|---|---|
| Mean | 98.7% | 98.5% | 99.1% |
| Acceptance Value | 5.5 | 7.9 | 5.5 |

The stability of these capsules were evaluated under accelerated and long term conditions. The stability results presented in Tables 6 (40° C./75% relative humidity (RH)), 7 (30° C./65% RH), and 8 (25° C./60% RH) confirm that the products showed minimal degradation under accelerated and long term stability conditions.

TABLE 6
Stress and Long Term Stability Results of Fingolimod
Capsules 0.5 mg; 40° C./75% RH

| | | | Any Other | |
|---|---|---|---|---|
| | | Acetyl | Unknown | Total |
| Batch No. | Conditions | Amino Diol | Impurity | Impurities |
| Lot No. | 1 M 40° C./75% RH | 0.05% | 0.08% | 0.13% |
| 1001046 | 3 M 40° C./75% RH | 0.12% | 0.17% | 0.36% |
| (1^{st} Batch) | 6 M 40° C./75% RH | 0.09% | 0.21% | 0.42% |
| Lot No. | 1 M 40° C./75% RH | 0.05% | 0.05% | 0.10% |
| 1001060 | 3 M 40° C./75% RH | 0.24% | <0.05% | 0.24% |
| (2^{nd} Batch) | 6 M 40° C./75% RH | 0.20% | 0.12% | 0.32% |
| Lot No. | 1 M 40° C./75% RH | 0.05% | 0.05% | 0.09% |
| 1001061 | 3 M 40° C./75% RH | 0.22% | 0.05% | 0.27% |
| (3^{rd} Batch) | 6 M 40° C./75% RH | 0.21% | 0.10% | 0.36% |

TABLE 7
Stress and Long Term Stability Results of Fingolimod
Capsules 0.5 mg; 30° C./65% RH

| | | Acetyl | Any Other | |
|---|---|---|---|---|
| | | Amino | Unknown | Total |
| Batch No. | Conditions | Diol | Impurity | Impurities |
| Lot No. | 3 M-30° C./65% RH | <0.05% | <0.05% | <0.05% |
| 1001046 | 6M-30° C./65% RH | <0.05% | 0.07% | 0.07% |
| (1^{st} Large | 9 M-30° C./65% RH | 0.07% | <0.05% | 0.07% |
| Batch) | 12 M-30° C./65% RH | 0.07% | 0.07% | 0.20% |
| Lot No. | 3 M-10° C./65% RH | 0.05% | <0.05% | 0.05% |
| 1001060 | 6 M-30° C./65% RH | 0.05% | <0.05% | 0.05% |
| (2^{nd} Batch) | 9 M-30° C./65% RH | 0.08% | <0.05% | 0.08% |
| | 12 M-30° C./65% RH | 0.08% | <0.05% | 0.08% |
| Lot No. | 3 M-30° C./65% RH | 0.05% | <0.05% | 0.05% |
| 1001061 | 6 M-30° C./65% RH | <0.05% | <0.05% | <0.05% |
| (3^{rd} Batch) | 9 M-30° C./65% RH | 0.08% | <0.05% | 0.08% |
| | 12 M-30° C./65% RH | 0.10% | <0.05% | 0.10% |

TABLE 8
Stress and Long Term Stability Results of Fingolimod
Capsules 0.5 mg; 25° C./60% RH

| | | Acetyl | Any Other | |
|---|---|---|---|---|
| | | Diol | Unknown | Total |
| Batch No. | Conditions | Impurity | Impurity | Impurities |
| Lot No. | 3 M-25° C./60% RH | <0.05% | <0.05% | <0.05% |
| 1001046 | 6 M-25° C./60% RH | <0.05% | <0.05% | <0.05% |
| (1^{st} Large | 9 M-25° C./60% RH | <0.05% | <0.05% | <0.05% |
| Batch) | 12 M-25° C./60% RH | <0.05% | <0.05% | <0.05% |
| Lot No. | 3 M-25° C./60% RH | 0.07% | <0.05% | 0.07% |
| 1001060 | 6 M-25° C./60% RH | <0.05% | 0.05% | 0.05% |
| (2^{nd} Batch) | 9 M-25° C./60% RH | <0.05% | <0.05% | <0.05% |
| | 12 M-25° C./60% RH | <0.05% | <0.05% | <0.05% |
| Lot No. | 3 M-25° C./60% RH | <0.05% | <0.05% | <0.05% |
| 1001061 | 6 M-25° C./60% RH | <0.05% | <0.05% | <0.05% |
| (3^{rd} Batch) | 9 M-25° C./60% RH | 0.05% | <0.05% | 0.05% |
| | 12 M-25° C./60% RH | <0.05% | <0.05% | <0.05% |

### Example 5

Using the dosage forms produced by Example 2, dissolution tests were conducted. Specifically, dosage forms were placed in baskets submerged in 500 milliliters of 0.1 N HCl with 0.2% sodium lauryl sulfate (as a surfactant). The solution was agitated at 100 RPM and the release of fingolimod hydrochloride from the dosage forms was assessed by HPLC method. Table 9 shows this data.

TABLE 9
In vitro dissolution profile of Fingolimod Hydrochloride
from dosage forms (500 mL, 0.1N HCl with 0.2% SLS at
37° C. ± 0.5° C., Apparatus 1 (baskets) at 100 rpm

| Agitation | % dissolution | | |
|---|---|---|---|
| Time | Lot#1001046 | Lot#1001060 | Lot#1001061 |
| 10 min. | Mean: 92% | Mean: 90% | Mean: 89% |
| | % RSD: 11.8 | % RSD: 7.9 | % RSD: 10.8 |
| 15 min. | Mean: 98% | Mean: 98% | Mean: 98% |
| | % RSD: 3.4 | % RSD: 4.0 | % RSD: 3.3 |
| 20 min. | Mean: 100% | Mean: 99% | Mean: 98% |
| | % RSD: 1.8 | % RSD: 3.8 | % RSD: 3.0% |
| 30 min. | Mean: 101% | Mean: 100% | Mean: 99% |
| | % RSD: 1.9 | % RSD: 3.5 | % RSD: 2.9% |

### Example 6

The bioequivalence of fingolimod capsules of the present invention (0.5 mg fingolimod; Lot#1001046) was compared to Novartis's GLENYA® capsules (also containing 0.5 mg fingolimod) following a single, oral 0.5 mg (1×0.5 mg) dose administration in healthy, adult, non-tobacco using male volunteers under fasting and fed conditions. The study was an open-label, single-dose, randomized, two-period, two-treatment crossover study. The content assay uniformity of the capsules (Lot. No 1001046) used in this bioequivalence study is presented in Table 10 below:

TABLE 10
Content Uniformity Testing Results (% Labeled Claim)

Lot #1001046

| | Beginning | Middle | End | Composite |
|---|---|---|---|---|
| | Sample | Sample | Sample | Sample |
| Mean | 101.3% | 99.2% | 100.4% | 99.5% |
| % RSD | 2.3% | 2.1% | 2.0% | 2.3% |

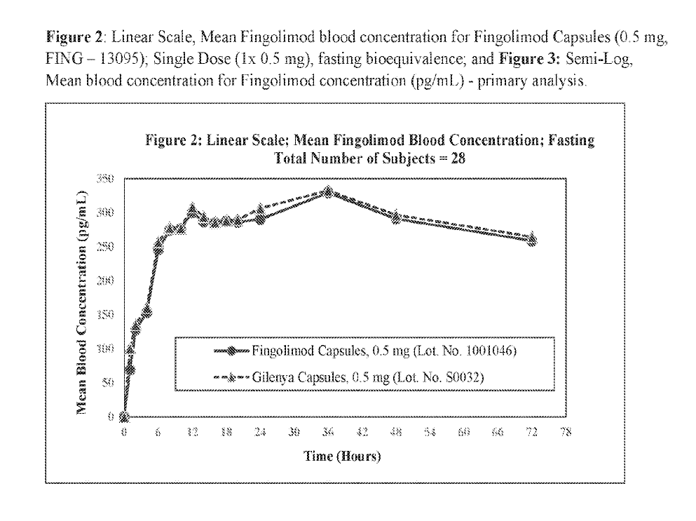

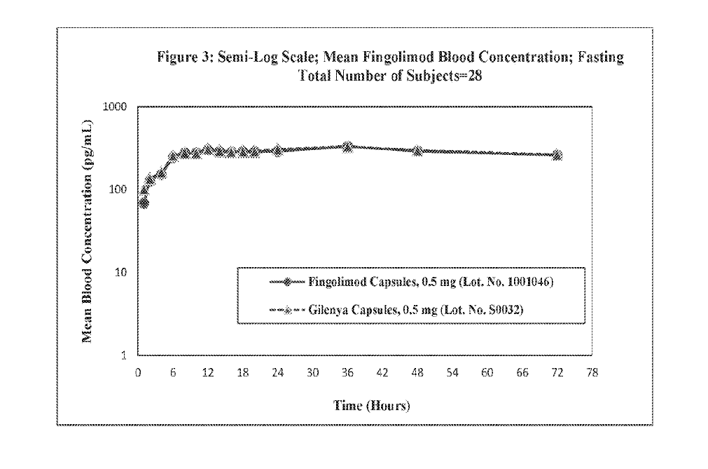

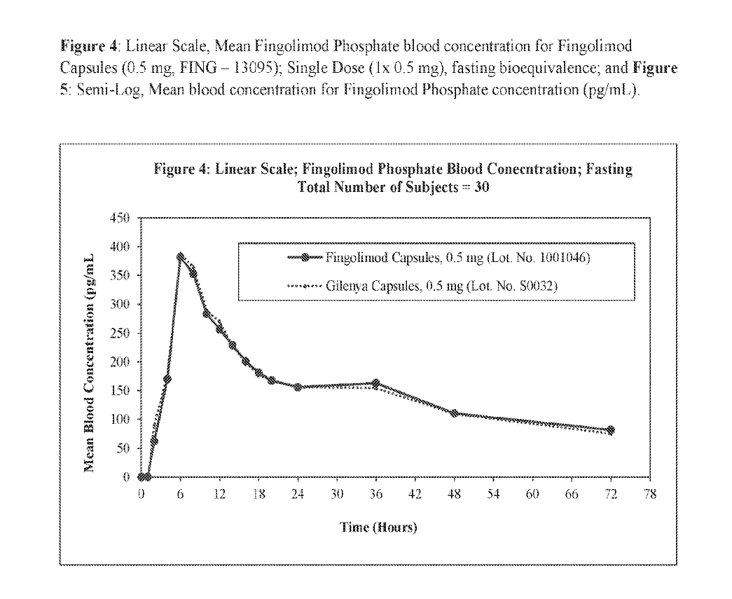

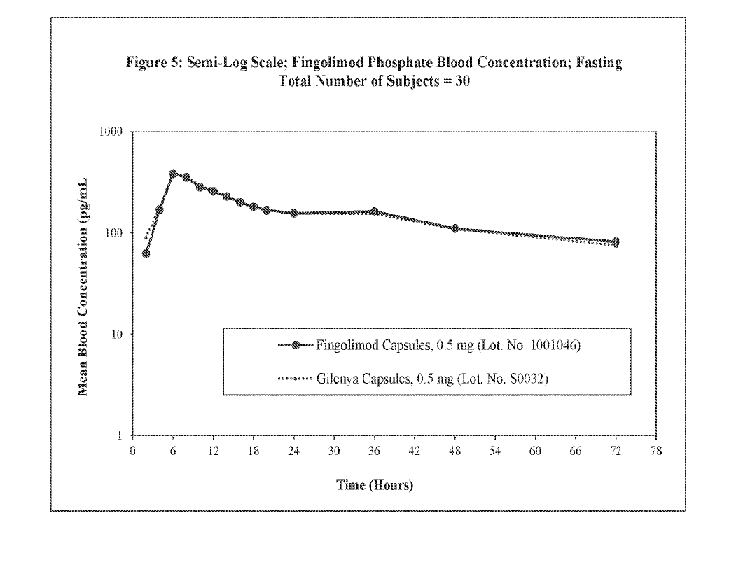

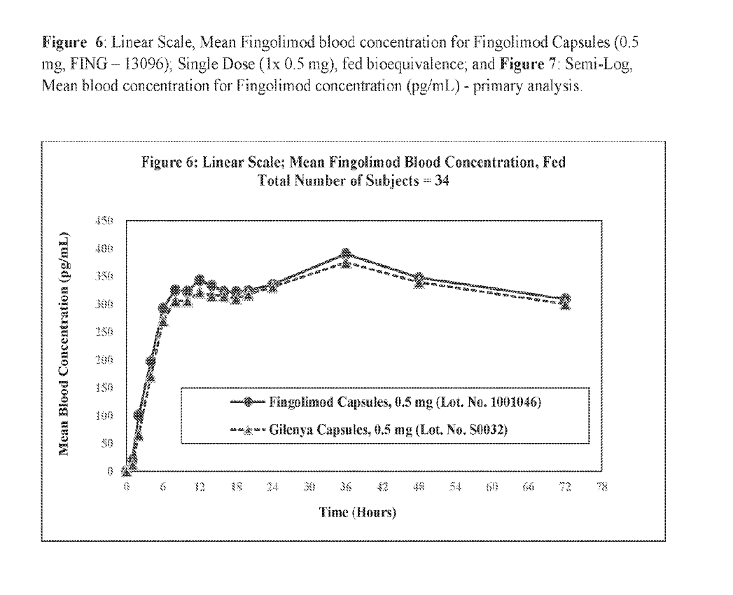

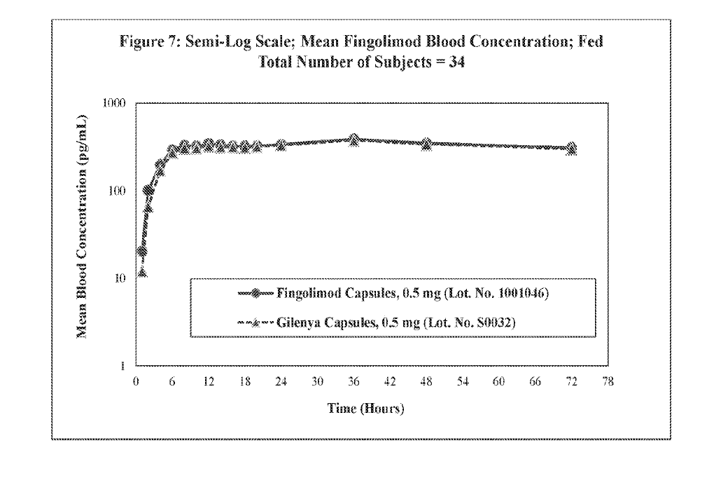

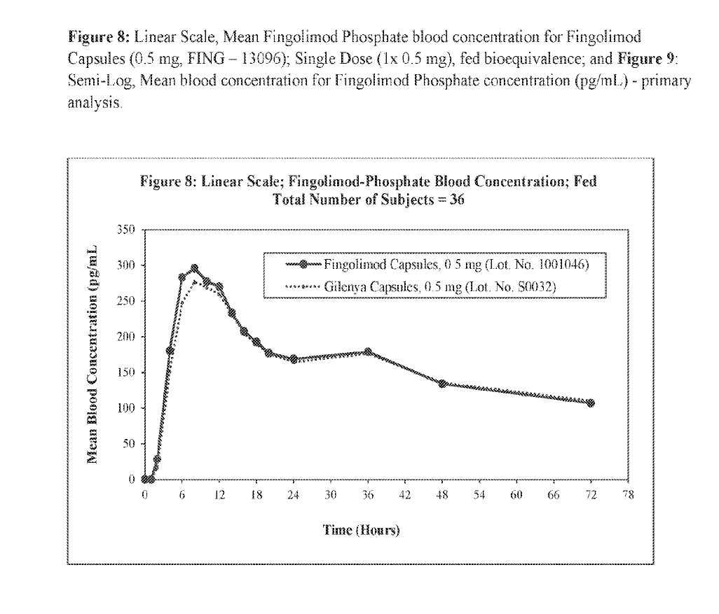

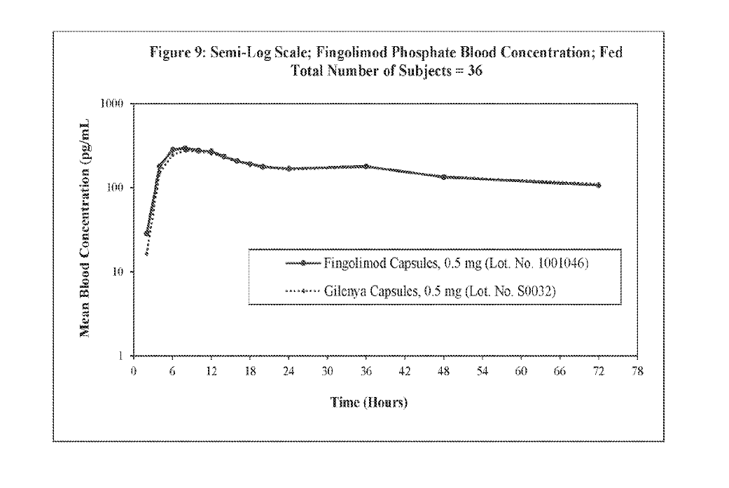

For the bioequivalence study, blood samples were collected within 120 minutes prior to dosing and at 1, 2, 4, 6, 8, 10, 12, 14, 16, 18, 20, 24, 36, 48 and 72 hours after dosing. The mean blood concentration versus time profile for the fingolimod formulation of the present invention compared to GILENYA® is illustrated graphically in FIGS. 2 to 9. Results with statistical analyses are also shown in tables A-D below. Single-dose pharmacokinetic parameters for fingolimod and fingolimod-phosphate were analyzed using ANOVA. Mean blood profiles are similar between the 0.5 mg fingolimod hydrochloride capsules of the present invention and the 0.5 mg fingolimod hydrochloride Novartis GILENYA® capsules. The statistical analyses of the fingolimod and fingolimod phosphate pharmacokinetic parameters are presented below. The maximum concentration (Cₘₐₓ) and the time at which it occurred relative to the administered dose (Tₘₐₓ) were determined from the observed blood concentration-time profile over the sampling time interval. Area under the curve from zero to seventy-two hours (AUC72) was the sum of the linear trapezoidal estimation of the areas from the time of dosing to 72 hours (the last blood sample collection). The primary pharmacokinetic variables for assessment of bioequivalence are CPEAK and AUC72 for fingolimod. Pharmacokinetic data from fingolimod-phosphate, the active metabolite of fingolimod is provided as supportive evidence of comparable therapeutic outcome. The 90% confidence intervals fall within 80%-125% for the test to reference ratio for the natural log transformed parameters. This study demonstrated that the formulation of 0.5 mg fingolimod capsule of the present invention are bioequivalent to Novartis' GILENYA® capsules following a single oral 0.5 mg (1×0.5 mg) dose administered under fasting and fed conditions.

TABLE A
Fingolimod Pharmacokinetic Parameters (Fasting Conditions)
Mean (% CV) Fingolimod Pharmacokinetic Parameters in Twenty-Eight
Healthy Male Subjects Following a Single Oral 0.5 mg (1 × 0.5 mg)
Dose of Fingolimod Capsules under Fasting Conditions
PROTOCOL NUMBER FING-13095

| | Arithmetic Mean | Arithmetic Mean | LSMEANS* | |
|---|---|---|---|---|
| Parameter | A = Mylan | B = GILENYA ® | Ratio (A/B) | 90% Confidence Interval** |
| AUCₙ (pg · hr/mL) | 20027 (20.19) | 20284 (22.29) | 1.00 | 95.77%-104.53% |
| CPEAK (pg/mL) | 337.5 (19.23) | 348.8 (21.22) | 0.98 | 93.37%-102.14% |
| TPEAK (hr) | 12.57 (44.88) | 10.64 (35.46) | | |

*Ratio (A/B) = e^{[LSMEAN of (LNA - LNB)]}
**Used Natural Log Transformed Parameter

TABLE B
Fingolimod Phosphate Pharmacokinetic Parameters (Fasting Conditions)
Mean (% CV) Fingolimod-Phosphate Pharmacokinetic Parameters in Thirty
Healthy Male Subjects Following a Single Oral 0.5 mg (1 × 0.5 mg)
Dose of Fingolimod Capsules under Fasting Conditions
PROTOCOL NUMBER FING-13095

| | Arithmetic Mean | Arithmetic Mean | LSMEANS* | |
|---|---|---|---|---|
| Parameter | A = Mylan | B = GILENYA ® | Ratio (A/B) | 90% Confidence Interval** |
| AUCₙ (pg · hr/mL) | 10905 (29.78) | 10808 (28.11) | 1.01 | 96.96%-104.68% |
| CPEAK (pg/mL) | 388.9 (25.51) | 395.4 (21.47) | 0.98 | 93.72%-101.82% |
| TPEAK (hr) | 6.600 (16.21) | 7.067 (20.67) | | |

*Ratio (A/B) = e^{[LSMEAN of (LNA - LNB)]}
**Used Natural Log Transformed Parameter

TABLE C
Fingolimod Pharmacokinetic Parameters (Fed conditions)
Mean (% CV) Fingolimod Pharmacokinetic Parameters in Thirty-Four
Healthy Male Subjects Following a Single Oral 0.5 mg (1 × 0.5 mg)
Dose of Fingolimod Capsules under Fed Conditions
PROTOCOL NUMBER FING-13096

| | Arithmetic Mean | Arithmetic Mean | LSMEANS* | |
|---|---|---|---|---|
| Parameter | A = Mylan | B = GILENYA ® | Ratio (A/B) | 90% Confidence Interval** |
| AUCₙ (pg · hr/mL) | 23385 (17.38) | 22341 (18.11) | 1.04 | 100.52%-107.73% |
| CPEAK (pg/mL) | 397.0 (15.39) | 382.5 (17.42) | 1.03 | 99.82%-106.99% |
| TPEAK (hr) | 32.54 (34.49) | 34.48 (31.86) | | |

*Ratio (A/B) = e^{[LSMEAN of (LNA - LNB)]}
**Used Natural Log Transformed Parameter

TABLE D
Fingolimod Phosphate (Metabolite) Pharmacokinetic Parameters (Fed conditions)
Mean (% CV) Fingolimod-Phosphate Pharmacokinetic Parameters in Thirty-Six
Healthy Male Subjects Following a Single Oral 0.5 mg (1 × 0.5 mg)
Dose of Fingolimod Capsules under Fed Conditions
PROTOCOL NUMBER FING-13096

| | Arithmetic Mean | Arithmetic Mean | LSMEANS* | |
|---|---|---|---|---|
| Parameter | A = Mylan | B = GILENYA ® | Ratio (A/B) | 90% Confidence Interval** |
| AUCₙ (pg · hr/mL) | 11639 (17.27) | 11326 (18.58) | 1.02 | 99.32%-105.33% |
| CPEAK (pg/mL) | 323.1 (17.58) | 298.3 (18.15) | 1.09 | 105.22%-112.87% |
| TPEAK (hr) | 7.778 (29.97) | 8.444 (27.80) | | |

*Ratio (A/B) = e^{[LSMEAN of (LNA - LNB)]}
**Used Natural Log Transformed Parameter

Nothing in the above description is meant to limit the present invention to any specific materials, geometry, or orientation of elements. Many modifications are contemplated within the scope of the present invention and will be apparent to those skilled in the art. The embodiments described herein were presented by way of example only and should not be used to limit the invention in any way.

1. A pharmaceutical dosage form, comprising:
a water-soluble filler;
a water-insoluble filler; and
fingolimod, wherein said pharmaceutical dosage form is substantially free of sugar alcohol.

2. The pharmaceutical dosage form of claim 1, wherein said water-soluble filler is selected from the group consisting of glycine, arginine, cysteine hydrochloride, methionine, and sodium chloride.

3. The pharmaceutical dosage form of claim 1, wherein said water-insoluble filler is selected from the group consisting of dibasic calcium phosphate dihydrate, anhydrous dibasic calcium phosphate, tribasic calcium phosphate, and calcium sulfate dihydrate.

4. The pharmaceutical dosage form of claim 1, wherein the water-soluble filler is glycine and the water-insoluble filler is dibasic calcium phosphate dihydrate.

5. The pharmaceutical dosage form of claim 4, wherein a concentration of said glycine is approximately equal to said concentration of said dibasic calcium phosphate dihydrate, by weight.

6. The pharmaceutical dosage form of claim 4, wherein glycine is present at a concentration about 5% to about 95%, by weight, and dibasic calcium phosphate dihydrate is present at a concentration about 5% to about 95%, by weight.

7. The pharmaceutical dosage form of claim 4, wherein said glycine is present at a concentration about 35% to about 49%, by weight.

8. The pharmaceutical dosage form of claim 4, wherein said dibasic calcium phosphate dihydrate is present at a concentration of about 35% to about 49%, by weight.

9. The pharmaceutical dosage form of claim 1, further comprising a lubricant and a glidant.

10. The pharmaceutical dosage form of claim 9, wherein the lubricant is selected from the group consisting of magnesium stearate, magnesium stearate with sodium lauryl sulfate (94:6), sodium stearyl fumarate, Compritol® 888 ATO, and calcium stearate.

11. The pharmaceutical dosage form of claim 9, wherein the glidant is colloidal silicon dioxide.

12. The pharmaceutical dosage form of claim 1, wherein the fingolimod is present at a concentration of 0.1 to 10 milligrams per pharmaceutical dosage form.

13. The pharmaceutical dosage form of claim 1, wherein said fingolimod is present as a pharmaceutically acceptable salt thereof.

14. The pharmaceutical dosage form of claim 13, wherein the pharmaceutically acceptable salt of fingolimod is fingolimod hydrochloride.

15. The pharmaceutical dosage form of claim 14, wherein the fingolimod hydrochloride is present in the pharmaceutical dosage form in an amount from about 0.2 mg to 5 mg.

16. The pharmaceutical dosage form of claim 14, wherein the fingolimod hydrochloride is present in the pharmaceutical dosage form in an amount from about 0.25 mg to 1 mg.

17. The pharmaceutical dosage form of claim 14, wherein the fingolimod hydrochloride is present in the pharmaceutical dosage form in an amount of 0.5 mg.

18. A method of making the pharmaceutical dosage form of claim 1 through use of mixing, dry granulation, wet granulation, or granulation by extrusion.

19. A pharmaceutical dosage form, comprising:
glycine present at 35% to 49%, by weight of the pharmaceutical dosage form;
dibasic calcium phosphate dihydrate present at 35% to 49%, by weight of the pharmaceutical dosage form; and
fingolimod hydrochloride, wherein said pharmaceutical dosage form is substantially free of sugar alcohol.

20. A process for producing a pharmaceutical dosage form, comprising the steps of:
a) combining fingolimod or a pharmaceutically acceptable salt thereof with a water-insoluble filler and a water-soluble filler to create a mixture;
b) passing the mixture through a roller compactor to form a compacted material;
c) milling the compacted material; and
c) blending the mixture with an extragranular excipient.

21. The method of claim 20, wherein the extragranular excipient is a lubricant, a glidant, or mixtures thereof.

22. The pharmaceutical dosage form of claim 4, wherein the pharmaceutical dosage form exhibits a bioavailability for fingolimod that is substantially similar to the bioavailability of fingolimod for GILENYA®.
